(19) United States
(12) Patent Application Publication
(10) Pub. No.: US 2017/0007582 A1
(43) Pub. Date: Jan. 12, 2017

(54) Use of Dipeptidyl Peptidase IV Effectors for Normalizing the Blood Glucose Level in Mammals

(71) Applicant: Royalty Pharma Collection Trust, New York, NY (US)

(72) Inventors: Hans-Ulrich Demuth, Halle (DE); Fred Rosche, Dieskau (DE); Joem Schmidt, Halle (DE); Robert P. Pauly, Vancouver (CA); Christopher H.S. McIntosh, Vancouver (CA); Ray A. Pederson, Vancouver (CA)

(21) Appl. No.: 15/042,892

(22) Filed: Feb. 12, 2016

Related U.S. Application Data
(63) Continuation of application No. 13/458,484, filed on Apr. 27, 2012, now abandoned.
Application No. 13/458,484 is a continuation of application No. 11/022,087, filed on Dec. 22, 2004, now abandoned.
Application No. 11/022,087 is a continuation of application No. 10/117,022, filed on Apr. 5, 2002, now abandoned.
Application No. 10/117,022 is a continuation of application No. 09/932,546, filed on Aug. 17, 2001, now abandoned.
Application No. 09/932,546 is a continuation-in-part of application No. 09/155,833, filed on Oct. 6, 1998, now Pat. No. 6,303,661.

(30) Foreign Application Priority Data
Apr. 25, 1996 (DE) 19616486.9

(51) Int. Cl. A61K 31/426; A61K 9/00

(52) U.S. Cl. CPC A61K 31/426; A61K 9/0053; A61K 9/0019

(57) ABSTRACT

The present invention comprises the use of activity-reducing effectors of dipeptidyl peptidase (DP IV) and DP IV-analogous enzyme activity in the blood of a mammal to lower elevated post-prandial and basal blood glucose levels in mammalian organisms.

The invention further comprises the use of activity-reducing effectors of dipeptidyl peptidase (DP IV) and DP IV-analogous enzyme activity in the blood of a mammal to increase the half-life of incretins in vivo.

### RELATED APPLICATIONS

This is a Continuation of application Ser. No. 09/932,546 filed Aug. 17, 2001, which is a Continuation-In-Part of application Ser. No. 09/155,833 filed Oct. 6 1998, which issued Oct. 16, 2001 as U.S. Pat. No. 6,303,661.

### FIELD OF THE INVENTION

The present invention relates to a novel method for the reduction in the concentration of circulating blood glucose and blood pressure by applying activity lowering effectors (substrates, pseudosubstrates, inhibitors, binding proteins, antibodies and the like) of enzymes with similar or identical activity to the enzymatic activity of the enzyme Dipeptidyl Peptidase TV.

### BACKGROUND OF THE INVENTION

Besides proteases involved in non-specific proteolysis, proteases resulting in the specific degradation of proteins are known which are involved in the functional regulation (activation, deactivation or modulation) of endogenous peptides. [KIRSCHKE, H., LANGNER, J., RIEMANN, S., WIEDERANDERS, B., ANSORGE, S. and BOHLEY, P., Lysosomal cysteine proteases. Excerpta Medica (Ciba Foundation Symposium 75), 15 (1980); KRÄUSSLICH, H.-G. and WIMMER, E., Viral Proteinases. Ann. Rev. Biochem. 57, 701 (1987)].

Such convertases, signal peptidases, or enkephalinases have been discovered in the immune system and as a result of neuropeptide research [GOMEZ, S., GLUSCHANKOF, P., LEPAGE, A., MARRAKCHI, N. and COHEN, P., Proc. Natl. Acad. Sci. USA 85, 5468 (1988); ANSORGE, S. and SCHÖN, E., Histochem. 82, 41 (1987)].

Since the amino acid proline, which is extraordinarily abundant in numerous peptide hormones, determines certain structural properties of these peptides, proline-specific peptidases have been discussed as having a similar function to the signal peptidases in the regulation of biologically active peptides. [YARON, A., The Role of Proline in the Proteolytic Regulation of Biologically Active Peptides. Biopolymers 26, 215 (1987); WALTER, R., SIMMONS, W. H. and YOSHIMOTO, T., Proline Specific Endo- and Exopeptidases. Mol. Cell. Biochem. 30, 111 (1980); VANHOOF, G., GOOSSENS, F., DE MEESTER, I., HENDRIKS, D. and SCHARPÉ, S., Proline motifs and their biological processing. FASEB Journal 9, 736 (1995)]. As a result of its exceptional structure, proline determines in such peptides both their conformation and stability, preventing degradation by non-specific proteases. [KESSLER, H., Conformation and is biological activity. Angew. Chem. 94, 509 (1982)]. In contrast, enzymes that are capable of highly specific actions on proline-containing sequences (including HIV-protease, cyclophylin, etc) are attractive targets of medicinal chemistry. In particular, the activity of post-proline-cleaving peptidases, such as Prolyl Endopeptidase (PEP) and Dipeptidyl Peptidase IV (DP IV), has been linked to the modulation of the biological activity of natural peptide substrates and their selective cleavage by these enzymes. It has been shown that PEP is involved in memory and learning, and that DP IV participates in signal transduction during the immune response [ISHIURA, S., TSUKAHARA, T., TABIRA, T., SHIMIZU, T., ARAHATA K. and SUGITA, H., FEBS-Letters 260, 131 (1990); HEGEN, M., NIEDOBITEK, G., KLEIN, C. E., STEIN, H. and FLEISCHER, B., J. of Immunology 144, 2908 (1990)].

In addition to their high proline specificity these enzymes are capable of selectively recognizing and cleaving peptide bonds containing the amino acid alanine in typical substrates. It is at present under discussion as to whether alanine-containing peptides adopt similar conformations to structurally related proline-containing peptides. Recently, such properties have been described by point mutation experiments involving the exchange of proline and alanine in proteins [DODGE, R. W. and SCHERAGA, H. A., Folding and unfolding kinetics of the proline-to-alanine mutants of bovine pancreatic ribonuclease A. Biochemistry 35 (5) 1548 (1996)].

DP IV or DP IV-like activity (i.e. the cytosolic DP II possesses almost identical substrate specificity to DP IV) present in the circulation is highly specific in releasing dipeptides from the N-terminal end of biologically active peptides with proline or alanine in the penultimate position of the N-terminal sequence of the peptide substrate. Hence, it has been concluded that this enzyme is involved in the regulation of the activity of polypeptides in vivo [VANHOOF, G., GOOSSENS, F., DE MEESTER, I., HENDRIKS, D. and SCHARPÉ, S., Proline motifs and their biological processing, FASEB Journal 9, 736 (1995)].

The glucose-dependent insulinotropic polypeptides: Gastric Inhibitory Polypeptide 1-42 (GIP₁₋₄₂) and Glucagon-Like Peptide Amide-1 7-36 (GLP-1₇₋₃₆), are hormones which potentiate glucose-induced insulin secretion from the pancreas (incretins), and are substrates of DP IV. The enzyme releases the dipeptides tyrosinyl-alanine and histidyl-alanine, respectively from the N-termini of these peptides both in vitro and in vivo. [MENTLEIN, R., GALLWITZ, B., and SCHMIDT, W. E., Dipeptidyl Peptidase IV hydrolyzes gastric inhibitory polypeptide, glucagon-like peptide-1 (7-36) amide, peptide histidine methionine and is responsible for their degradation in human serum. Eur. J. Biochem. 214, 829 (1993)].

Reduction in the cleavage of such substrates by DP IV or DP IV-like enzyme activity in vivo can serve to effectively suppress undesirable enzymatic activity under both laboratory conditions and in pathological states in mammals [DEMUTH, H.-U., Recent developments in the irreversible inhibition of serine and cysteine proteases. J. Enzyme Inhibition 3, 249-278 (1990); DEMUTH, H.-U. and HEINS, J., On the catalytic Mechanism of Dipeptidyl Peptidase IV. in Dipeptidyl Peptidase IV (CD 26) in Metabolism and the Immune Response (B. Fleischer, Ed.) R. G. Landes, Biomedical Publishers, Georgetown, 1-35 (1995)]. For instance, non-insulin dependent Diabetes mellitus is associated with insulin resistance and insulin secretion which is inappropriate for the prevailing glucose concentration, and which may be partially related to protease-mediated abnormalities in the concentration of circulating incretins [BROWN, J. C., DAHL, M., KWAWK, S., MCINTOSH, C. H. S., OTTE, S. C. and PEDERSON, R. A. Peptides 2, 241 (1981); SCHMIDT, W. E., SIEGEL, E. G., GALLWITZ, B. KUMMEL, H., EBERT, R. and CREUTZFELDT, W., Characterization of the insulinotropic activity of fragments derived from gastric inhibitory polypeptide. Diabetologia 29, 591A (1986); ADELHORST, K., HEDEGAARD, B. B., KNUDSEN, L. B. and KIRK, O., Structure-activity studies of glucagon-like peptide. J. Biol. Chem. 296, 6275 (1994)].

Insulin-dependent Diabetes mellitus (IDDM) is currently treated through the administration of insulin (isolated from bovine or porcine pancreases or produced as a recombinant molecule) to patients using different forms of administration. Non-insulin-dependent Diabetes mellitus (NIDDM) is treated by diet, administration of sulphonylureas to stimulate insulin secretion or with biguanides to increase glucose uptake. Resistant individuals may need insulin therapy. Traditional, as well as more modern, methods for the treatment of IDDM are characterized by a great deal of effort on behalf of the patient, high costs, and usually a drastic reduction in the quality of living of the patient. Standard therapy (daily i.v. injection of insulin), which has been used since the thirties, is directed at treating the acute symptoms but results, after prolonged application, in vascular disease and nerve damage [LACY, P., Status of Islet Cell Transplantation. Diabetes Care 16 (3) 76 (1993)]. More modern methods, such as the installation of subcutaneous depot—implants (insulin release occurring free from proteolytic attack and in small doses, without the need of daily injections) as well as implantation (or transplantation) of intact islet of Langerhans cells are under trial. However, such transplantation is expensive. Additionally, they represent risky surgical intervention and require, in the case of transplantation methods, immunsupression or bypassing the immune response. [LACY, P., Treating Diabetes with Transplanted Cells. Sci. Americ. 273 (1) 40-46 (1995)]. Attempts at reducing glucose disposal have not been successful. In the case of NIDDM, many patients treated by stimulating endogenous insulin secretion with sulphonylureas become resistant to these drugs. In addition, increasing glucose disposal with biguanides has met with limited success.

In contrast to the above therapies, the suggested administration of highly effective, low-molecular weight enzyme inhibitors represents a cost-effective alternative. Such inhibitors of various proteolytic enzymes are already in use as anti-hypertensive drugs, immunosuppressive drugs, and antiviral agents. Chemical design of molecules with consideration to their stability, transport and clearance properties may be used to modify their efficacy, and even to adapt the compounds to individual differences between organisms. [SANDLER, M. and SMITH, H. J., eds., Design of Enzyme Inhibitors as Drugs. Oxford University Press, Oxford (1989); MUNROE, J. E., SHEPHERD, T. A., JUNGHEIM, L. N., HORNBACK, W. J., HATCH, S. D., MUESING, M. A., WISKERCHEN, M. A., SU, K. S., CAMPANALE, K. M., BAXTER, A. J., and COLACINO, J. M., Potent, orally bioavailable HIV-1 protease inhibitors containing noncoded D-amino acids. Bioorg. Medicinal Chem. Letters 5 (23) 2897 (1995)].

### SUMMARY OF INVENTION

The present invention relates to a novel method in which reduction of the activity of the enzyme Dipeptidyl Peptidase (DP IV or CD 26), or of DP IV-like enzyme activity, in the blood of mammals by specific enzyme effectors will result in a reduced degradation of the endogenous, or exogenously administrated, insulinotropic peptides (incretins), Gastric Inhibitory Polypeptide/Glucose-dependent Insulinotropic Polypeptide 1-42 (GIP₁₋₄₂) and Glucagon-like Peptide-1 7-36 amide (GLP-1₇₋₃₆) (or analogs of these peptides). The decrease in concentration of these peptides or their analogs, resulting from degradation by DP IV and DP IV-like enzymes, will be thus be reduced or delayed.

As a consequence of the enhanced stability of the endogenous, or exogenously administered, incretins or their analogs, caused by a reduction in DP IV-activity, their insulinotropic effects are enhanced, resulting in a potentate stimulation of insulin secretion from the pancreatic islets of Langerhans, and more rapid removal of glucose from the blood. As a result, glucose tolerance is improved.

As a consequence, metabolic abnormalities associated with Diabetes mellitus, including abnormalities of carbohydrate and lipid metabolism, glucosuria and severe metabolic acidosis, and chronic alterations such as microvascular and macrovascular disease and polyneuropathy, which are the consequence of prolonged, elevated circulating glucose concentrations, are prevented or alleviated and in particular blood pressure levels are reduced.

The present invention is a new approach to lowering elevated concentrations of blood glucose. It is simple, commercially useful, and is suitable to be used in the therapy, especially of human diseases, which are caused by elevated or extraordinary blood glucose and/or blood pressure levels.

### BRIEF DESCRIPTION OF DRAWINGS

Further understanding of the present invention may be had by reference to the accompanying drawings wherein:

FIG. 1 shows MALDI-TOF -analysis of the DP IV-catalyzed hydrolysis of GIP₁₄₂ (a) and GLP-₇₋₃₆ and their inhibition by isoleucyl thiazolidine (b).

FIG. 2 shows HPLC-analysis of the serum presence of GLP-1 metabolites in presence of the DP IV inhibitor isoleucyl thiazolidine in vivo.

FIG. 3 shows influence of the DP IV-inhibitor isoleucyl thiazolidine on different blood parameter of the i.d.-glucose-stimulated rat.

FIG. 4 shows influence of chronic oral treatment of fatty (fa/fa) VDF Zucker rats by the DP IV-inhibitor isoleucyl thiazolidine on the fasting blood glucose during 12 weeks of drug application.

FIG. 5 Influence of chronic treatment of fatty (fa/fa) VDF Zucker rats by the DP. IV-inhibitor isoleucyl thiazolidine on the systolic blood pressure within 8 weeks of to drug application (systolic blood pressure was measured using the tail-cuff procedure).

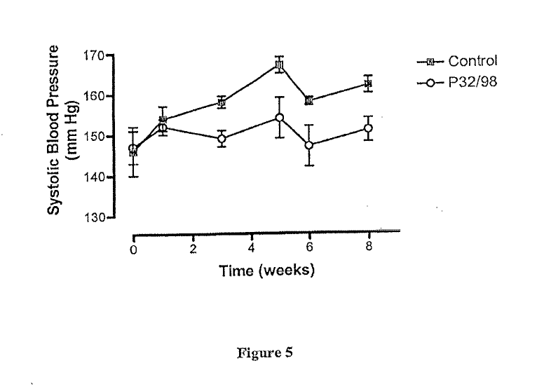

### DETAILED DESCRIPTION OF THE INVENTION

The aim of the present invention is a simple and new method to lower the level of blood glucose and/or blood pressure in which reduction in the activity of the enzyme Dipeptidyl Peptidase (DP IV or CD 26) or of DP IV-like enzyme activity in the blood of mammals induced by effectors of the enzyme will lead to a reduced degradation of the endogenous (or exogenously administrated) insulinotropic peptides Gastric Inhibitory Polypeptide 1-42 (GIP₁₋₄₂) and Glucagon-Like Peptide Amide-1 7-36 (GLP-1₇₋₃₆) (or analogs of these peptides). The decrease in concentration of these peptides or their analogs, normally resulting from degradation by DP IV and DP IV-like enzymes, will thus be reduced or delayed.

The present invention is based on the striking finding that a reduction in the circulating enzymatic activity of Dipeptidyl Peptidase (DP IV or CD 26) or of DP IV-like enzyme activity in the blood of mammals results in an improved glucose tolerance.

We observed that:
1. Reduction of Dipeptidyl Peptidase (DP IV or CD 26) or of DP IV-like enzyme activity leads to a relative increase in the stability of glucose-stimulated endogenously released or exogenously administrated incretins (or their analogs) with the consequence that the administration of effectors of DP IV or of DP IV-like proteins can be used to control the incretin degradation in the circulation.
2. The enhanced biological stability of the incretins (or their analogs) results in a modification of the insulin response.
3. The enhanced stability of the circulating incretins, caused by reduction of Dipeptidyl Peptidase (DP IV or CD 26) or of DP IV-like enzyme, results in subsequent modification of insulin-induced glucose disposal, indicating that glucose tolerance can be improved by applying DP IV-effectors.
4. Blood pressure levels can be reduced.

Accordingly, the invention concerns the use of effectors of Dipeptidyl Peptidase (DP IV) or of DP IV-like enzyme activity, for lowering of elevated blood glucose and/or blood pressure levels, such as those found in mammals demonstrating clinically inappropriate basal and post-prandial hyperglycemia. The use according to the invention is more specifically characterized by the administration of effectors of DP IV or of DP IV-analogous enzyme activity in the prevention or alleviation of pathological abnormalities of Metabolism of mammals such as glucosuria, hyperlipidaemia, m+etabolic acidosis and diabetes mellitus. In a further preferred embodiment, the invention concerns a method of lowering elevated blood glucose levels in mammals. Such as those found in a mammal demonstrating clinically inappropriate basal and post-prandial hyperglycemia, comprising administering to a mammal in need of such treatment a therapeutically effective amount of an effector of Dipeptidyl Peptidase (DP IV) or of DP IV-like enzyme activity.

In another preferred embodiment, the invention concerns effectors of Dipeptidyl Peptidase (DP IV) or of DP IV-like enzyme activity for use in a method of lowering elevated blood glucose and/or blood pressure levels in mammals, such as those found in mammals demonstrating clinically inappropriate basal and post-prandial hyperglycemia.

The administered effectors of DP IV and DP IV-like enzymes according to this invention may be employed in pharmaceutical formulations as enzyme inhibitors, substrates, pseudosubstrates, inhibitors of DP IV gene expression, binding proteins or antibodies of the target enzyme proteins or as a combination of such different compounds, which reduce DP IV and DP IV-like protein concentration or enzyme activity in mammals. Effectors according to the invention are, for instance, DP IV-inhibitors such as dipeptide derivatives or dipeptide mimetics as alanyl pyrolidide, isoleucyl thiazolidine as well as the pseudosubstrate N-valyl prolyl, O-benzoyl hydroxylamin. Such compounds are known from the literature [DEMUTH, H.-U., Recent developments in the irreversible inhibition of serine and cysteine proteases. J. Enzyme Inhibition 3, 249 (1990)] or may be synthesized according to methods described in the literature.

The method according to the present invention is a new approach to the reduction of elevated circulating glucose concentration in the blood of mammals and to reducing blood pressure levels.

The method is simple, commercially useful and appropriate for use in therapy, especially of human diseases, which are caused by elevated or inappropriate blood glucose levels.

The effectors are administrated in the form of pharmaceutical preparations containing the effector in combination with state-of-the-art materials for drug delivery. The effectors are administered either parenterally (i.v. in physiological saline solution) or enterally oral, formulated with usual carrier materials, like e.g., glucose.

Depending on the endogenous stability and on the bioavailibility of the effectors single or multiple administrations are suitable, to reach the anticipated normalization of the blood glucose concentration. Such dosage range may vary from 0.1 mg to 10.0 mg of effector compound per kilogram, e.g. in the case of the aminoacyl thiazolidines as inhibitors of DP IV.

### EXAMPLES

### Example 1

### Inhibition of the DP IV-Catalyzed Hydrolysis of the Incretins GIP₁₋₄₂ and GLP-1₇₋₃₆ In Vivo

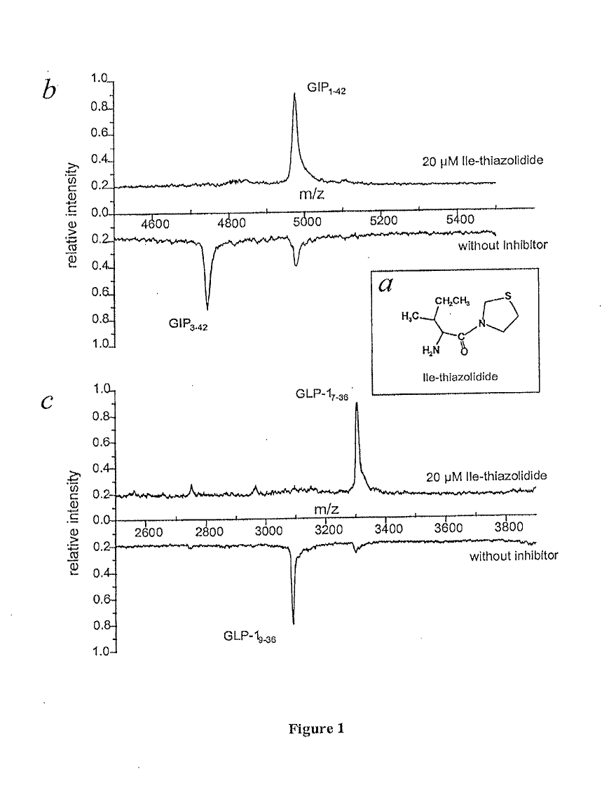

It is possible to suppress the in vitro hydrolysis of incretins caused by DP IV and DP IV-like enzymatic activity using purified enzyme or pooled human serum (FIG. 1).

According to the present invention complete suppression of the enzyme-catalyzed hydrolysis of both peptide hormones is achieved in vitro by incubating 30 mM GIP₁₋₄₂ or 30 mM GLP-1₇₋₃₆ and 20 mM isoleucyl thiazolidine (1a), a reversible DP IV-inhibitor in 20% of pooled serum at pH 7.6 and 30° C. over 24 hours (1b and 1c, both upper spectra: Synthetic GIP₁₋₄₂ (5 mM) and synthetic GLP-1₇₋₃₆ (15 μM) were incubated with human serum (20%) in 0.1 mM TRICINE Puffer at pH 7.6 and 30° C. for 24 hours. Samples of the incubation assays (in the case of GIP₁₋₄₂ 2.5 pmol and in the case of GLP-1₇₋₃₆ 7.5 pmol) have been withdrawn after different time intervals. Samples were cocrystallized using 2′,6′-dihydroxyacetophenon as matrix and analyzed by MALDI-TOF-mass spectrometry. Spectra (FIG. 1) display accumulations of 250 single laser shots per sample.

(1b) The signal of m/z 4980.1±5.3 corresponds to the DP IV-substrate GIP₁₋₄₂ (M 4975.6) and the signal of the mass m/z 4745.2±5.5 corresponds to the DP IV-released product GIP3-42 (M 4740.4).

(1c) The signal of m/z 3325.0±1.2 corresponds to the DP IV-substrate GLP-1₇₋₃₆ (M 3297.7) and the signal of mass m/z 3116.7±1.3 to the DP IV-released product GLP-1₉₋₃₆ (M 3089.6).

In the control assays containing no inhibitor the incretins were almost completely degraded (FIGS. 1b and 1c, both bottom spectra).

### Example 2

### Inhibition of the Degradation of GLP1₇₋₃₆ by the DP IV-Inhibitor Isoleucyl Thiazolidine In Vivo

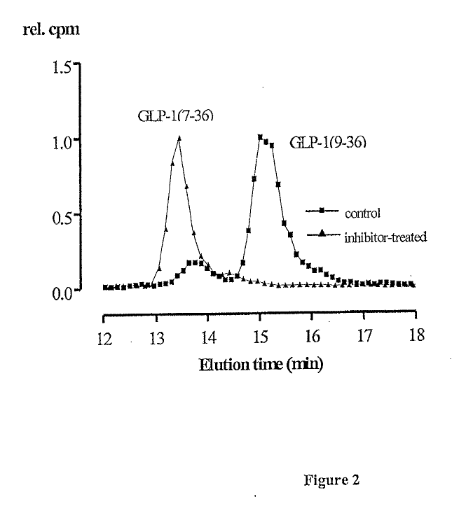

Analysis of the metabolism of native incretins (in this case GLP-1₇₋₃₆) in the circulation of the rat in the presence or absence of the DP IV-inhibitor isoleucyl thiazolidine (i.v. injection of 1.5 M inhibitor in 0.9% saline solution) and of a control. No degradation of the insulinotropic peptide hormone GLP-1₇₋₃₆ occurs at a concentration of 0.1 mg/kg of the inhibitor isoleucyl thiazolidine in treated animals (n=5) during the time course of the experiment (FIG. 2).

To analyze the metabolites of the incretins in the presence and absence of the DP IV-inhibitor, test and control animals received a further i.v. injection of 50-100 pM ¹²⁵I-GLP-1₇₋₃₆ (specific activity about 1 μCi/pM) 20 min after an initial i.v.-inhibitor and/or saline administration. Blood samples were collected after 2-5 min incubation time and the plasma was extracted using 20% acetonitrile. Subsequently, the peptide extract was separated on RP-HPLC. Multiple fractions of eluent were collected between 12-18 min and counted on a γ-counter. Data are expressed as counts per minute (cpm) relative to the maximum.

### Example 3

### Modulation of Insulin Responses and Reduction of the Blood Glucose Level After i.v. Administration of the DP IV-Inhibitor Isoleucyl Thiazolidine In Vivo

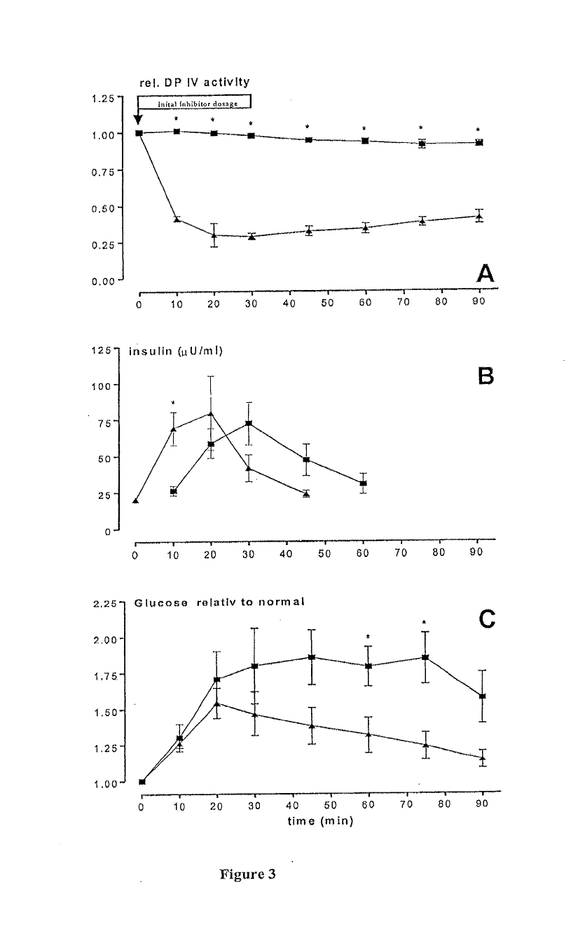

The figure shows circulating glucose and insulin responses to intraduodenal (i.d.) administration of glucose to rats in the presence or absence of isoleucyl thiazolidine (0.1 mg per kg). There is a more rapid reduction in the circulating glucose concentration in animals, which received DP IV-effectors when compared to untreated controls. The observed effect is dose dependent and reversible after termination of an infusion of 0.05 mg/min of the DP IV-inhibitor isoleucyl thiazolidine per kg rat. In contrast to the i.d. glucose-stimulated animals, there was no comparable effect observable after the i.v. administration of the same amount of glucose in inhibitor-treated control animals. In FIG. 3 these relationships are demonstrated displaying the inhibitor-dependent changes of selected plasma parameter: A—DP IV-activity, B—plasma-insulin level, C—blood glucose level.

### Example 4

### Impact of Chronic Treatment of Fatty Zucker Rats on the Fasting Blood Glucose During 12 Weeks of Drug Application

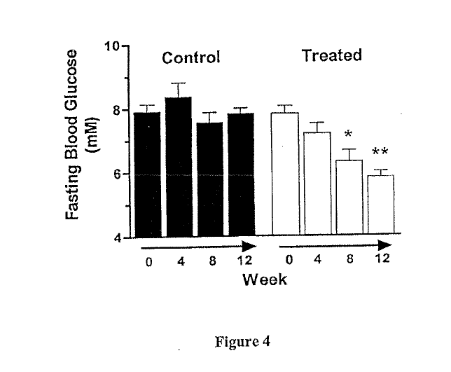

Chronic application of the DP IV-inhibitor isoleucyl thiazolidine fumarate results in dramatic reduction and almost normalization of the fasting blood glucose in the chosen diabetic rat model (FIG. 4).

Animals. Six pairs of male fatty (fa/fa) VDF Zucker rat littermates were randomly assigned to either a control or treatment (isoleucyl thiazolidine fumarate) group at 440 g body weight (11±0.5 weeks of age). Animals were housed singly, on a 12 hour light/dark cycle (lights on at 6 am) and allowed access to standard rat food, and water ad libitum.

Protocol for daily monitoring and drug administration. The treatment group received 10 mg/kg isoleucyl thiazolidine fumarate by oral gavage twice daily (8:00 a.m. and 5:00 p.m.) for 100 days, while the control animals received concurrent doses of vehicle consisting of a 1% cellulose solution. Every two days, body weight, morning and evening blood glucose, and food and water intake were assessed. Blood samples for glucose determination were acquired from tail bleeds, and measured using a SureStep glucose analyzer (Lifescan Canada Ltd., Burnaby).

Protocol for monthly assessment of glucose tolerance. Every four weeks from the start of the experiment, an oral glucose tolerance test (OGTT) was performed: animals were fasted for 18 hours following the 1700h dosing and administered 1 g/kg glucose orally. This time period is equivalent to ˜12 circulating half-lives of isoleucyl thiazolidine fumarate.

### Example 5

### Impact of Chronic Treatment of Fatty Zucker Rats on Systolic Blood Pressure with the DP IV-Inhibitor Isoleucyl Thiazolidine

Chronic application of the DP IV-inhibitor isoleucyl thiazolidine fumarate results in the stabilization of systolic blood pressure in the chosen diabetic rat model (FIG. 4).

Animals. Six pairs of male fatty (fa/fa) VDF Zucker rat littermates were randomly assigned to either a control or treatment (isoleucyl thiazolidine fumarate) group at 440 g body weight (11±0.5 weeks of age). Animals were housed singly, on a 12 hour light/dark cycle (lights on at 6 am) and allowed access to standard rat food, and water ad libitum.

Protocol for daily monitoring and drug administration. The treatment group received 10 mg/kg isoleucyl thiazolidine fumarate by oral gavage twice daily (8:00 a.m. and 5:00 p.m.) for 100 days, while the control animals received concurrent doses of vehicle consisting of a 1% cellulose solution. Systolic blood pressure was measured weekly using the tail-cuff procedure.

The test animals (n=5, male Wistar-rats, 200-225 g) initially received 1.5 M Isoleucyl-Thiazolidine in 0.9% saline solution (^{▴}) or the same volume of plain 0.9% saline solution (^{▪}) (control group n=5). The test group additionally obtained an infusion of the inhibitor of 0.75 M/min over 30 min experimental time (*). The control group received during the same time interval an infusion of inhibitor-free 0.9% saline solution. At starting time t=0 all animals were administered an i.d. glucose dose of 1 g/kg 40% dextrose solution (w/v). Blood samples were collected of all test animals in 10 min time intervals. Glucose was analyzed using whole blood (Lifescan One Touch II analyzer) while DP IV-activity and insulin concentration were analyzed in plasma. The insulin radioimmunoassay was sensitive over that range 10 and 160 mU/ml [PEDERSON, R. A., BUCHAN, A. M. J., ZAHEDI-ASH, S., CHEN, C. B. & BROWN, J. C. Reg. Peptides. 3, 53-63 (1982)]. DP IV-activity was estimated spectrophotometrically [DEMUTH, H.-U. and HEINS, J., On the catalytic Mechanism of Dipeptidyl Peptidase IV. in Dipeptidyl Peptidase IV (CD 26) in Metabolism and the Immune Response (B. Fleischer, Ed.) R. G. Landes, Biomedical Publishers, Georgetown, 1-35 (1995)]. All data are presented as mean+/−s.e.m.

1.-7. (canceled)

8. A method for lowering elevated fasting blood glucose levels in mammals comprising administering a therapeutically effective amount of at least one inhibitor of Dipeptidyl Peptidase (DP IV) or enzymes having DP IV-like enzyme activity.

9. The method according to claim 8, wherein said at least one inhibitor is administered orally in combination with at least one carrier substance.

10. The method according to claim 8, wherein said at least one inhibitor is administered in multiple administrations.

11. The method according to claim 8, wherein the mammals demonstrate clinically inappropriate basal and post-prandial hyperglycemia.

12. The method according to claim 8, wherein the administration is for the prevention or alleviation of pathological abnormalities of metabolism of mammals such as glucosuria, hyperlipidaemia, metabolic acidosis and Diabetes mellitus.

13. (canceled)
